(19) United States
(12) Patent Application Publication
(10) Pub. No.: US 2025/0288508 A1
(43) Pub. Date: Sep. 18, 2025

(54) NOVEL TRANSPARENT COSMETIC COMPOSITION FOR SKIN AND HAIR HYGIENE AND CARE

(71) Applicant: PIERRE FABRE DERMO-COSMETIQUE, LAVAUR (FR)

(72) Inventors: Laetitia LEMERCIER, CASTRES (FR); Céline ALIAGA, CASTRES (FR); Anne-Laure GAUDRY, CASTRES (FR)

(73) Assignee: PIERRE FABRE DERMO-COSMETIQUE, LAVAUR (FR)

(21) Appl. No.: 18/860,261

(22) PCT Filed: Apr. 21, 2023
(86) PCT No.: PCT/EP2023/060431

§ 371 Date: Oct. 25, 2024

(30) Foreign Application Priority Data
Apr. 28, 2022 (FR) FR2203993

(51) Int. Cl. A61K 8/44; A61K 8/60; A61K 8/73; A61Q 5/02; A61Q 19/10

(52) U.S. Cl. CPC A61K 8/44; A61K 8/602; A61K 8/73; A61Q 5/02; A61Q 19/10; A61K 2800/262

(57) ABSTRACT

The invention relates to a transparent cosmetic composition containing a mixture of surfactants comprising a single anionic surfactant selected from alaninates, acyl glutamic acids and the cosmetically acceptable salts thereof, the nonionic surfactant lauryl glucoside alone or accompanied by at least one nonionic surfactant selected from alkyl glucosides in which the alkyl radical comprises 8 to 16 carbon atoms, 1.5% to 2.5% by weight of at least one amphoteric surfactant other than amidopropyl betaines, at least 65% by weight of water relative to the total weight of the composition, and at least one cosmetically acceptable excipient. This composition is free of amphoteric surfactant from the class of amidopropyl betaines and surfactant comprising at least one sulfate group. It is particularly suitable for skin and hair hygiene and care.

### TECHNICAL FIELD OF THE INVENTION

The present invention relates to a novel transparent cosmetic composition and to the use of such a composition for washing and/or care of the skin and/or hair. The invention also relates to a cosmetic treatment method for skin and/or hair hygiene and/or care using such a composition.

### PRIOR ART

A cosmetic composition intended for skin and/or hair washing and/or care must be effective, in other words cleansing, foaming and conditioning. More specifically, the main expectations of consumers are to have products which produce a lot of foam and which have a conditioning effect for the hair. Furthermore, such a composition must comprise the least possible or be devoid of any controversial substance, because consumers are increasingly vigilant regarding the composition of their cosmetic products. Thus, they are looking for products free from chemical substances, preferring ingredients of natural origin, known for their better tolerance by and affinity with the skin, and which are more respectful of the environment.

It is also preferable that such a cosmetic composition is transparent, because this conveys a feeling and a guarantee of safety for many consumers.

Furthermore, such a cosmetic composition can be deployed in a plurality of packagings. These may be tubes or pump dispensers, which brings about constraints in terms of the release of the product. This specificity generally leads inventors to give the composition certain values of viscosity. A formula that is too thick will not be pumpable, and a formula that is too fluid will escape from the consumers hands, which does not correspond to the organoleptic quality for hygiene products.

Many cosmetic compositions for body and hair applications proposed by the prior art contain sulfated surfactants. These surfactants ensure a particularly interesting foaming effect, giving the impression of gentle cleansing. However, in reality they are very irritating to the skin, desiccating and allergenic.

Similarly, the presence of some amphoteric surfactants such as amidopropyl betaines, and in particular cocamidopropyl betaine, can provoke certain intolerances such as skin irritations. It has been shown that two impurities found in these amidopropyl betaines are responsible for this bad press. These are 3,3-dimethylaminopropylamine (DMAPA) and amidoamine, which are known for their irritating and sensitizing properties (Fowler et al. Contact Dermatitis 37, 276-281, 1997; Angelini et al. Contact Dermatitis 34, 248-252, 1996).

Thus, for good skin tolerance, these ingredients must be used in low concentrations, but still always with a risk when addressing consumers with particularly marked skin sensitivity.

The present invention aims to overcome the disadvantages of the cosmetic compositions proposed by the prior art, in particular the disadvantages outlined above, by proposing a transparent cosmetic composition which contains as few as possible, or which does not contain any, ingredients which are not of natural origin, which has a physiological pH, which respects the epidermis and thus has the least possible risk of skin intolerance, while having a foaming power that is at least as good as, or even better than, that of the cosmetic compositions of the prior art.

Additional objectives of the invention are that this composition contains the least possible ingredients, and that it is suitable for use on all skins, including sensitive skin and atopic skin.

Another aim of the invention is that this transparent composition has a sufficient viscosity so as not to escape from the user's hand when it is handled and enabling its distribution by various types of packaging, in particular tubes and pump dispensers.

### DISCLOSURE OF THE INVENTION

The present inventors have discovered that such objectives can be attained by using a particular combination of surfactants. This combination has, unexpectedly, a particularly high foaming power, a good viscosity, and can form transparent cosmetic compositions with reduced risk of skin irritation compared with the cosmetic compositions of the prior art.

A “good viscosity” of a composition is intended to mean a viscosity enabling said composition to be dispensed by various packagings, in particular tubes and pump dispensers, and enabling it not to escape from the consumers hands when it is dispensed.

According to a first aspect, the invention proposes a transparent cosmetic composition which is suitable, in particular, for a topical application, in particular on the skin and hair. This transparent composition contains:
a mixture of surfactants comprising:
   at least one anionic surfactant selected from alaninates, acyl glutamic acids and the cosmetically acceptable salts thereof,
   the non-ionic surfactant lauryl glucoside, alone or accompanied by at least one non-ionic surfactant selected from alkyl glucosides in which the alkyl radical comprises 8 to 16 carbon atoms,
   at least one amphoteric surfactant other than amidopropyl betaines, and
at least one cosmetically acceptable excipient.

In addition, this transparent cosmetic composition preferably comprises at least 50% by weight of water, relative to the total weight of the composition, and it is free of amphoteric surfactants from the class of amidopropyl betaines and surfactants comprising at least one sulfate group.

In the present description, the term “cosmetically acceptable” is intended to mean that the salt or excipient is suitable for use by contacting it with human and animal cells, in particular skin or hair cells. Preferably, the excipient has an agreeable odor, color and feel, and does not generate unacceptable discomforts which could divert a user from the composition according to the invention.

A transparent composition is defined in the present description, in conventional manner, as a composition having a turbidity less than 20 NTU. Turbidity is measured using a HACH TL2310 turbidity meter. Before each turbidity measurement, correct adjustment of the instrument is checked by measuring the turbidity of two standards from a StabCal 0 to 4000 NTU standards calibration kit, the standards being chosen so that they frame the expected value of the sample.

In the present description, the term “at least one” means a single compound or a mixture of several such compounds. Thus, the mixture of surfactants of the composition according to the invention contains:
a single anionic surfactant selected from alaninates, acyl glutamic acids and the cosmetically acceptable salts thereof, or a plurality of such anionic surfactants;
a single non-ionic surfactant which is lauryl glucoside, or a plurality of non-ionic surfactants including lauryl glucoside as well as one or more non-ionic surfactants selected from alkyl glucosides in which the alkyl radical comprises 8 to 16 carbon atoms;
a single amphoteric surfactant other than amidopropyl betaines, or a plurality of such amphoteric surfactants other than amidopropyl betaines.

Such a specific mixture of surfactants advantageously ensures a high foaming power of the transparent cosmetic composition, as well as good quality of the foam formed. Without assuming the phenomena underlying this performance of the surfactants mixture according to the invention, it may be thought to be due to a synergistic effect of the particular groups carried by these surfactants. This performance is advantageously obtained in the absence in the composition of any amidopropyl betaine type amphoteric surfactant conventionally used in cosmetic compositions for hygiene or care of the skin and hair, which have the disadvantage of a risk of skin irritation. The transparent composition obtained therefore presents no risk of skin irritation, or a greatly reduced risk compared with the compositions of the prior art. Moreover, said mixture of surfactants gives the transparent cosmetic composition a good viscosity and, in particular, a viscosity enabling said composition to be devoid of thickening agent, and this even when the composition comprises one or more cosmetically acceptable excipients such as emollients, conditioners, solubilizers and perfumes.

In the present invention, the term “amidopropyl betaines” is intended to mean the following compounds: behenamidopropyl betaine, capryl/capramidopropyl betaine, cocamidopropyl betaine, isostearamidopropyl betaine, lauramidopropyl betaine, myristamidopropyl betaine, stearamidopropyl betaine, undecyleneamidopropyl betaine, and amidopropyl betaines derived from: almond, apricot, avocado, babassu, rapeseed, coconut oil, sunflower oil, oat, olive, palm oil, castor oil, soybean, shea butter and wheat germ.

In addition, the transparent cosmetic composition according to the invention is not in the form of an emulsion comprising an aqueous phase and a fatty phase, or a two-phase composition, but in the form of a single-phase fluid composition.

In particular embodiments, the invention also responds to the following features, implemented separately or in each of their technically operable combinations.

According to a particular embodiment of the present invention, the transparent cosmetic composition according to the invention does not include any additional ingredient selected from fatty substances. According to another particular embodiment, the transparent composition includes a total quantity of additional ingredient(s) selected from fatty substances less than or equal to 2% by weight, preferably less than or equal to 1% by weight, relative to the total weight of the composition. In particular embodiments of the invention, the transparent cosmetic composition contains a total content of additional ingredient(s) selected from fatty substances between 0.001 and 2% by weight, in particular between 0.001 and 1% by weight, more particularly between 0.001 and 0.5% by weight, and in particular between 0.001 and 0.2% by weight, relative to the total weight of the composition. The total content(s) of additional ingredient(s) selected from fatty substances indicated above are such that they enable the transparent cosmetic composition of the present invention to keep a form which is not that of an emulsion comprising an aqueous phase and a fatty phase.

Within the meaning of the present invention, a “fatty substance” means an organic compound that is insoluble in water at 25° C., 1 atm, in other words with solubility less than 5% by weight, preferably less than 1% by weight. Fatty substances are generally soluble in organic solvents under the same conditions of temperature and pressure, for example in chloroform, ethanol, benzene or petroleum jelly oil.

The expression “fatty substance” encompasses, in particular, fatty alcohols, fatty acid esters, fatty acids, fatty amides, mineral oils or waxes (or hydrocarbon), animal oils or waxes or plant oils or waxes, etc.

According to a particular embodiment of the present invention, the transparent cosmetic composition does not include any emulsifier. It is, in particular, free of emulsifiers such as a fatty acid monoester of polyethylene glycol.

According to a particular embodiment of the present invention, the transparent composition which is an object of the present invention has a turbidity less than 20 NTU for at least 12 months after its preparation. Preferably, the transparent composition has a turbidity less than 20 NTU for at least 24 months after its preparation, and yet more preferably it has a turbidity less than 20 NTU for at least 36 months after its preparation.

According to a particular embodiment of the present invention, the one or more anionic surfactants selected from alaninates are preferably selected from sodium cocoyl alaninate or trisodium dicarboxymethyl alaninate.

According to a particular embodiment of the present invention, the one or more anionic surfactants selected from acyl glutamic acids and the cosmetically acceptable salts thereof are preferably selected from acyl glutamic acids in which the acyl group comprises a saturated or unsaturated, linear or branched, hydrocarbon chain, with 8 to 30 carbon atoms, preferably with 10 to 20 carbon atoms and more preferably with 12 to 18 carbon atoms. The acyl group can, in particular, be selected from the lauroyl, myristoyl, palmitoyl, stearoyl, olivoyl, cocoyl and oleoyl groups. The cosmetically acceptable salt of the acyl glutamic acid can be selected from alkali metal salts, alkaline earth metal salts and ammonium salts. It is for example selected from the sodium, potassium, lithium or magnesium salts, sodium and potassium salts being particularly preferred in the context of the invention.

According to a particular embodiment of the present invention, the one or more anionic surfactants selected from acyl glutamic acids and the cosmetically acceptable salts thereof can be, for example, chosen in the group consisting of compounds with INCI names sodium cocoyl glutamate, disodium cocoyl glutamate, sodium myristoyl glutamate, sodium lauroyl glutamate, potassium lauroyl glutamate, potassium cocoyl glutamate and sodium stearoyl glutamate. Several anionic surfactants selected from acyl glutamic acids and the cosmetically acceptable salts thereof can be used in the composition according to the invention, which can, for example, contain any mixture of two or more of the compounds mentioned above.

Preferably, the composition according to the invention comprises a single anionic surfactant selected from alaninates. More preferably, the composition according to the invention comprises a single anionic surfactant, sodium cocoyl alaninate.

According to a particular embodiment of the present invention, the transparent composition preferably comprises a single non-ionic surfactant, lauryl glucoside.

According to another particular embodiment of the invention, the transparent composition comprises at least one second non-ionic surfactant selected from alkyl glucosides in which the alkyl radical comprises 8 to 16 carbon atoms. The alkyl glucosides are preferably selected from the compounds with INCI names: octyl glucoside and decyl glucoside.

According to a particular embodiment of the present invention, the one or more amphoteric surfactant(s) other than amidopropyl betaines are selected from Sodium Caproamphoacetate, Sodium Caproamphopropionate, Sodium apryloamphoacetate, Sodium Capryloamphopropionate, Sodium Cocoamphoacetate, Sodium Cocoamphopropionate, Sodium Isostearoamphoacetate, Sodium Isostearoamphopropionate, Sodium Lauroamphoacetate, Sodium Lauroamphopropionate, Sodium Oleoamphoacetate, Sodium Oleoamphopropionate, Sodium Stearoamphoacetate, Stearoamphopropionate, Sodium Taîlowamphoacetate, Sodium heatgermamphoacetate, Disodium Caproamphodiacetate, Disodium Caproamphodipropionate, Disodium Capryloamphodiacetate, Disodium Cocoamphodiacetate, Disodium Isostearoamphodiacetate, Disodium Lauroamphodiacetate, Disodium Oleoamphodipropionate, Disodium Capryloamphodipropionate, Disodium Cocoamphodipropionate, Disodium Isostearoamphodipropionate, Disodium Lauroamphodipropionate, Disodium Stearoamphodiacetate, Tallowamphodiacetate and Disodium Disodium Wheatgermamphodiacetate.

Preferably, said at least one amphoteric surfactant other than amidopropyl betaines is selected from lauryl betaine, coco-betaine, sodium cocoamphoacetate and disodium cocoamphodiacetate, or the mixtures thereof.

Preferably, said at least one amphoteric surfactant other than amidopropyl betaines is selected from lauryl betaine, coco-betaine and sodium cocoamphoacetate, or the mixtures thereof.

According to a particular embodiment of the present invention, the transparent composition comprises at least 50% by weight of water, relative to the total weight of the composition. Preferably, the composition according to the invention comprises at least 55% by weight of water, relative to the total weight of the composition. Preferably, the composition according to the invention comprises at least 60% by weight of water relative to the total weight of the composition, and more preferably, the composition according to the invention comprises at least 65% by weight of water relative to the total weight of the composition. Preferably, the transparent cosmetic composition according to the invention comprises at least 70% by weight of water relative to the total weight of the composition.

In particular embodiments of the present invention, the transparent cosmetic composition is free of ethylenediaminetetraacetic acid (EDTA).

According to a particular embodiment of the present invention, the transparent composition according to the invention is free of any surfactant of the type with an isethionic acid or isethionate group, in particular surfactant selected from acyl isethionic acids or their salts, such as the substance with INCI name sodium cocoyl isethionate. Without altering the performance of the transparent composition, this advantageously reduces the presence within said composition, of compounds conventionally used in cosmetic compositions for hygiene or care of the skin and hair, which have the disadvantage of a non-natural origin.

According to a particular embodiment of the present invention, the transparent composition is free of any surfactant of the type with a sulfosuccinic acid or sulfosuccinate group, in particular surfactant selected from the alkyl sulfosuccinic acids or their salts, such as the substance with INCI name lauryl sulfosuccinate. Without altering the performance of the transparent composition, this also advantageously reduces the presence within said composition, of compounds conventionally used in cosmetic compositions for hygiene or care of the skin and hair, which have the disadvantage of a non-natural origin.

According to a particular embodiment of the present invention, the transparent composition is free of surfactant comprising at least one sulfate group, in other words an —O—SO3H or —O—SO2—O— group, such as laureth sulfate or lauryl sulfate. Without altering the performance of the transparent composition, this advantageously reduces the presence within said composition, of compounds conventionally used in cosmetic compositions for hygiene or care of the skin and hair, which have the disadvantage of a non-natural origin. Moreover, although sulfated surfactants have a particularly interesting foaming power, they are aggressive for the epidermis. Thus, their absence in the transparent composition that is the object of the present invention advantageously reduces the risks of aggression of the user's epidermis.

More generally, the transparent composition according to the invention preferably contains the fewest possible surfactants. Thus, in particular embodiments of the invention, the transparent composition does not comprise more than 6 surfactants, preferably not more than 5 surfactants, more preferably not more than 4 surfactants and preferably 3 surfactants. As previously indicated, a high foaming power as well as the other advantages cited can be obtained with a mixture of surfactants comprising a single compound from each of three defined families of surfactants (one anionic surfactant selected from alaninates and/or acyl glutamic acids and the cosmetically acceptable salts thereof, the non-ionic surfactant lauryl glucoside and one amphoteric surfactant other than amidopropyl betaines), advantageously making it possible to minimize the number of ingredients present in the composition and thus to reduce the cost of production of the composition.

In preferred embodiments of the invention, the content of said mixture of surfactants is between 5 and 18% by weight, preferably between 6 and 16% by weight, more preferably between 8 and 15% by weight, relative to the total weight of the transparent composition. Such ranges of values make it possible to obtain a good foaming power of the composition, a good quality of foam formed and a good viscosity of the composition, while being associated with a reduced production cost of the composition.

The total content of said at least one anionic surfactant selected from alaninates or acyl glutamic acids and the cosmetically acceptable salts thereof, is preferably between 1.5 and 5% by weight, preferably between 2 and 4% by weight, relative to the total weight of the composition.

The total content of lauryl glucoside as sole non-ionic surfactant of the composition is preferably between 3 and 10% by weight, preferably between 5 and 8% by weight, relative to the total weight of the composition.

In a particular embodiment, the total content of non-ionic surfactants consisting of lauryl glucoside accompanied by at least one non-ionic surfactant selected from alkyl glucosides in which the alkyl radical comprises 8 to 16 carbon atoms is preferably between 3 and 10% by weight, preferably between 5 and 8% by weight, relative to the total weight of the composition.

The total content of said at least one amphoteric surfactant other than amidopropyl betaines is preferably between 0.5 and 3% by weight, more preferably between 1.5 and 2.5% by weight, relative to the total weight of the composition.

According to a preferred embodiment of the present invention, the transparent cosmetic composition contains:
a mixture of surfactants comprising:
   a single anionic surfactant selected from alaninates, acyl glutamic acids and the cosmetically acceptable salts thereof,
   the non-ionic surfactant lauryl glucoside, alone or accompanied by at least one non-ionic surfactant selected from alkyl glucosides in which the alkyl radical comprises 8 to 16 carbon atoms,
   1.5% to 2.5% by weight of at least one amphoteric surfactant other than amidopropyl betaines, relative to the total weight of the composition, and
at least one cosmetically acceptable excipient,
said composition comprising at least 65% by weight of water relative to the total weight of the composition, the content of said mixture of surfactants being between 5 and 18% by weight relative to the total weight of the composition, and said composition being free of amphoteric surfactants from the class of amidopropyl betaines and surfactants comprising at least one sulfate group.

In particular embodiments of the present invention, the transparent composition has a viscosity greater than 800 cPs, preferably a viscosity between 800 and 5500 cPs.

In a particular embodiment of the invention, the transparent composition is free of thickeners, in order to minimize the number of ingredients in the formulas. As previously explained, the mixture of surfactants used in the transparent composition advantageously gives this composition a good viscosity and, in particular, a viscosity enabling the composition to be devoid of thickening agent and this even when the composition comprises one or more cosmetically acceptable excipients such as emollients, conditioners, solubilizers, and perfumes. The number of ingredients of the composition is thus reduced and the associated production costs are therefore also reduced.

In other particular embodiments of the present invention, the composition nevertheless comprises at least one thickening agent.

Within the meaning of the present invention, the term “thickening agent” preferably means alginates, carrageenans, cellulose, starch, gums selected from: diutan gum, gellan gum, sclerotium gum, succinoglycan gum, gum arabic, locust bean gum, Tamarindus indica seed gum, and caesalpinia spinosa gum.

In particular embodiments of the invention, said at least one cosmetically acceptable excipient is selected from emollients, conditioners, solubilizers, perfumes, preservatives and the mixtures thereof.

In particular embodiments of the present invention, the transparent composition contains one or more emollients, in particular selected from glycerol, diglycerol or sorbitol.

In particular embodiments of the present invention, the transparent composition contains one or more conditioners, in particular selected from guars, polyquaterniums, polyglyceryls, polysaccharides, squalane etc. Preferably, the conditioner is a guar. According to a preferred embodiment of the present invention, the total content of conditioner(s) is between 0.1 and 1% by weight relative to the total weight of the transparent composition.

In particular embodiments of the present invention, the transparent composition according to the invention contains one or more solubilizers, in particular selected from the family of polyglyceryls, such as polyglyceryl-4 caprate, polyglyceryl-6 caprylate, polyglyceryl-3 cocoate, polyglyceryl-6 ricinoleate, glyceryl oleate, coco-glucoside.

In particular embodiments of the present invention, the transparent composition contains one or more cosmetically active ingredients, in particular by topical application, for example selected from the hydrating agents, antioxidants, vitamins, etc.

The total content of cosmetically active ingredient(s) in the transparent composition according to the invention is preferably between 0.05 and 3% by weight, relative to the total weight of the composition.

In particular embodiments of the invention, the transparent composition comprises one or more cosmetically active ingredient(s), selected from hyaluronic acid, oat sap, helychrysum extracts, helychrysum essential oils, limnanthe oil, in particular Limnanthes alba seed oil, oat seedlings, flower acids, such as the product marketed under the name Acides de Fleurs®, Garcinia extracts, milk thistle oil, niacinamide, Edelweiss extracts, such as the product marketed under the name Alpaflor® Edelweiss, quinquina extracts, caffeine, peony extracts, mango butter and citron extracts, such a list not limiting the invention in any way.

The transparent composition according to the invention, in its desired form, can be prepared by any conventional method per se for the preparation of cosmetic compositions. A particularly preferred preparation method in the context of the invention is the successive introduction into a mixer, at low speed, of each of the surfactants in the water of the formula, then optional addition of other excipients and/or active ingredients and finally stirring/homogenization of the formula. The pH of the formula thus obtained can also be adjusted, if necessary.

In the particular embodiments in which the transparent cosmetic composition comprises at least one conditioner, this conditioner is preferably introduced beforehand under vigorous stirring into the water of the formula.

The transparent composition that is the object of the present invention can be used on all keratinous materials, such as the skin, scalp, hair, eyelashes, eyebrows, nails or mucous membranes, in particular packaged in the form of a hygiene product, for example in the form of a product for cleaning the skin, mucous membranes and/or hair, in particular in the form of a product for cleaning and/or removing make-up from the skin, in particular from the face and/or the body, in the form of a shower product, in the form of a shampoo.

Another aspect of the invention relates to the non-therapeutic cosmetic use of a transparent cosmetic composition according to the invention for washing and/or care of the skin, in particular the skin of the body and/or face and/or scalp, and/or the hair, of an individual, preferably by topical application.

This use preferably comprises successive steps consisting of:
applying the transparent composition on the skin and/or hair, the latter having preferably been made wet beforehand,
developing foam, in particular by rubbing the skin and/or hair on which the composition has been applied,
and rinsing, preferably with water, optionally after a resting time and/or an optional massage.

These steps can, for example, be implemented once or twice per day, for example morning and evening, or as often as necessary.

The transparent composition according to the invention can, in particular, be used for washing and/or the care of sensitive or atopic skin.

In the present description, sensitive skin means a skin that is subject to irritations or allergic reactions because of various internal and external non-immunological factors. The term atopic skin means a skin that is subject to an inflammatory disease characterized by a dryness of the skin associated with itching such as, for example, in the case of eczema or atopic dermatitis.

The invention also relates to a non-therapeutic cosmetic treatment method for hygiene and/or care of the skin and/or hair of an individual, by means of a transparent cosmetic composition according to the invention. This method comprises successive steps consisting of:
applying the transparent composition on the skin and/or hair, the latter having preferably been made wet beforehand,
developing foam by rubbing the composition on the skin and/or the hair,
and rinsing, preferably with water.

This method can respond to any feature, or combination of features, described above with reference to the cosmetic use of the composition according to the invention.

The features and advantages of the invention will become more apparent in light of the examples of implementation below, provided purely by way of illustrative and non-limiting examples of the invention.

### EXAMPLES

### 1/Example of Composition

A transparent cosmetic composition according to the invention, intended for cosmetic treatment, for hygiene and care, of hair, has the characteristics listed in table 1 below:

TABLE 1

| Ingredient (INCI name) | % wt./wt. |
|---|---|
| Sodium cocoyl alaninate | 3-4 |
| Lauryl glucoside | 5-7 |
| Coco betaine | 1.5-2.0 |
| Guar hydroxypropyltrimonium chloride | 0.1-0.5 |
| Water | Qs |
| Sodium benzoate | 0.45 |
| Active ingredients | 1 |
| Perfume | <0.5 |
| Aloe Vera | <0.1 |

### Table 1—Example of Transparent Composition According to the Invention for Hygiene and Care of Hair

Another example of a transparent composition according to the invention, intended for cosmetic treatment, for hygiene and care, of the skin of the face and/or of the body, is given in table 2 below:

TABLE 2

| Ingredient (INCI name) | % wt./wt. |
|---|---|
| Sodium cocoyl alaninate | 3-4 |
| Lauryl glucoside | 5-7 |
| Coco betaine | 1.5-2.0 |
| Water | Qs |
| Sodium benzoate | 0.45 |
| Active ingredients | 1 |
| Perfume | <0.5 |

Table 2—Example of a Transparent Composition According to the Invention for Hygiene and Care of the Skin of the Face and/or Body

### 2/Evaluation of the Characteristics of the Compositions According to the Invention

Transparent compositions are evaluated according to the following criteria:
Foaming power (quantity/quality): foam height, foam creaminess, foam softness,
Viscosity,
pH: the measurement is made with a pH meter
Stability over time,
Organoleptic characteristics (color, odor)

For this purpose, the following operating protocols are implemented.

For the evaluation of the foam height, a 250-ml graduated measuring test tube, of outer diameter 38.9 cm and height 36 cm, with a stopper, is used. 0.5 g of a composition is placed in this test tube and demineralized water is added to make up to 50 ml. The test tube is stoppered then vigorously shaken 10 times. The foam height is read and expressed in mm.

The results obtained with various anionic surfactants are indicated in table 3 below:

TABLE 3

| | Composition | Composition | Composition |
|---|---|---|---|
| | 1 | 2 | 3 |
| Ingredient (INCI name) | % wt./wt. | % wt./wt. | % wt./wt. |
| Sodium cocoyl alaninate | 3-4 | — | — |
| Sodium cocoyl glutamate | — | 3-4 | — |
| Disodium cocoyl glutamate | — | — | 3-4 |
| Lauryl glucoside | 5-7 | 5-7 | 5-7 |
| Decyl glucoside | — | — | — |
| Coco glucoside | — | — | — |
| Coco betaine | 1.5-2.0 | 1.5-2.0 | 1.5-2.0 |
| Lauryl betaine | — | — | — |
| Cocoamphoacetate | — | — | — |
| Cocamidopropyl betaine | — | — | — |
| Guar | — | — | — |
| Water | Qs | Qs | Qs |
| Sodium benzoate | 0.45 | 0.45 | 0.45 |
| Citric acid monohydrate | 0.68 | 0.98 | 0.98 |
| Foam height (mm) | 210 | 180 | 190 |
| Viscosity (cPs) | 2164 | 3681 | 3404 |
| pH | 5.35 | 4.75 | 5.21 |
| Turbidity (NTU) | 5.56 | 5.06 | 6.42 |

### Table 3—Evaluation of Various Criteria for Transparent Compositions According to the Invention as a Function of Different Selected Anionic Surfactants

It can be seen that compositions 1, 2 and 3 have a very interesting foam height and an expected viscosity. It is interesting to note that the largest height is obtained with sodium cocoyl alaninate, moreover the foam appears particularly creamy.

The results obtained with various non-ionic surfactants are indicated in table 4 below:

TABLE 4

| | | Composition | |
|---|---|---|---|
| | Composition 4 | 5 | Composition 6 |
| Ingredient (INCI name) | % wt./wt. | % wt./wt. | % wt./wt. |
| Sodium cocoyl alaninate | 3-4 | 3-4 | 3-4 |
| Sodium cocoyl glutamate | — | — | — |
| Disodium cocoyl | — | — | — |
| glutamate | | | |
| Lauryl glucoside | 5-7 | — | — |
| Decyl glucoside | — | — | 5-7 |
| Coco glucoside | — | 5-7 | — |
| Coco betaine | 1.5-2.0 | 1.5-2.0 | 1.5-2.0 |
| Lauryl betaine | — | — | — |
| Cocoamphoacetate | — | — | — |
| Cocamidopropyl betaine | — | — | — |
| Guar | 0.1-0.5 | 0.1-0.5 | 0.1-0.5 |
| Water | Qs | Qs | Qs |
| Sodium benzoate | 0.45 | 0.45 | 0.45 |
| Citric acid monohydrate | 0.67 | 0.67 | 0.67 |
| Foam height (mm) | 190 | — | — |
| Viscosity (cPs) | 3500 | 679 | 520 |
| pH | 5.33 | 5.0 | 4.8 |
| Turbidity (NTU) | 6.78 | — | — |

### Table 4—Evaluation of Various Criteria for Transparent Compositions According to the Invention (Composition 4) and Not According to the Invention (Compositions 5 and 6), as a Function of Different Selected Non-Ionic Surfactants

Thus, it can be seen that in the case where a single non-ionic surfactant is used to produce a transparent composition according to the invention, only lauryl glucoside enables a composition according to the invention (composition 4) to be obtained, in other words a composition with a certain viscosity, in particular a viscosity greater than 800 cPs. Indeed, in the presence of coco-glucoside or decyl glucoside as the only non-ionic surfactant, the compositions prove to be too liquid (compositions 5 and 6). Thus no foam height could be measured.

The results obtained with various amphoteric surfactants are indicated in table 5 below:

TABLE 5

| | Composition | Composition | Composition | Composition |
|---|---|---|---|---|
| | 4 | 7 | 8 | 9 |
| Ingredient (INCI | % wt./wt. | % wt./wt. | % wt./wt. | % wt./wt. |
| name) | | | | |
| Sodium cocoyl | 3-4 | 3-4 | 3-4 | 3-4 |
| alaninate | | | | |
| Sodium cocoyl | — | — | — | — |
| glutamate | | | | |
| Disodium cocoyl | — | — | — | — |
| glutamate | | | | |
| Lauryl glucoside | 5-7 | 5-7 | 5-7 | 5-7 |
| Decyl glucoside | — | — | — | — |
| Coco glucoside | — | — | — | — |
| Coco betaine | 1.5-2.0 | — | — | — |
| Lauryl betaine | — | 1.5-2.0 | — | — |
| Cocoamphoacetate | — | — | 1.5-2.0 | — |
| Cocamidopropyl | — | — | — | 1.5-2.0 |
| betaine | | | | |
| Guar | 0.1-0.5 | 0.1-0.5 | 0.1-0.5 | 0.1-0.5 |
| Water | Qs | Qs | Qs | Qs |
| Sodium benzoate | 0.45 | 0.45 | 0.45 | 0.45 |
| Citric acid | 0.67 | 0.60 | 0.89 | 0.62 |
| monohydrate | | | | |
| Foam height (mm) | 190 | 190 | 190 | 190 |
| Viscosity (cPs) | 3500 | 2700 | 2323 | 2892 |
| pH | 5.33 | 5.36 | 5.37 | 5.37 |
| Turbidity (NTU) | 6.78 | 6.91 | 9.64 | 7.43 |

### Table 5—Evaluation of Various Criteria for Transparent Compositions According to the Invention as a Function of Different Selected Amphoteric Surfactants

It can be seen that all the compositions 4, 7, 8 and 9 have a similar foam height. The amphoteric surfactant does not appear to influence the foaming power.

### 3/Comparative Tests with a Composition from the Prior Art

The foam height and viscosity of the composition marketed under the name PLANTAPON® SF by BASF comprising sodium cocoamphoacetate, glycerol, lauryl glucoside, sodium cocoyl glutamate and sodium lauryl glucose carboxylate, have been evaluated.

The composition PLANTAPON® SF is tested under the same conditions of total surfactant concentration as the transparent cosmetic composition that is the object of the present invention.

For this purpose, the same operating protocols as in point 2/above are implemented.

In other words, for the evaluation of the foam height, a 250-ml graduated measuring test tube, of outer diameter 38.9 cm and height 36 cm, with a stopper, is used. 0.5 g of the composition PLANTAPON® SF is placed in the test tube and demineralized water is added to make up to 50 ml. The measured viscosity of the composition is 1158 cPs. The test tube is stoppered then vigorously shaken 10 times. The foam height is read and expressed in mm. The foam height obtained is 180 mL.

In conclusion, the transparent cosmetic composition that is the object of the present invention which comprises only a single anionic surfactant selected from alaninates, acyl glutamic acids and the cosmetically acceptable salts thereof, has a foaming power as good as if not better than the composition PLANTAPON® SF comprising two anionic surfactants (sodium cocoyl glutamate and lauryl glucose carboxylate). The transparent cosmetic composition of the present invention is therefore just as effective as, if not more effective than, PLANTAPON SF and, above all, it is more economical because said effectiveness is achieved with fewer ingredients.

More generally, it should be noted that the implementations and embodiments of the invention considered hereinabove have been described as non-limiting examples, and that other variants could consequently be considered.

1. A transparent cosmetic composition which contains:
a mixture of surfactants comprising:
a single anionic surfactant selected in the group consisting of alaninates, acyl glutamic acids and the cosmetically acceptable salts thereof,
the non-ionic surfactant lauryl glucoside, alone or accompanied by at least one non-ionic surfactant selected in the group consisting of alkyl glucosides in which the alkyl radical comprises 8 to 16 carbon atoms,
1.5% to 2.5% by weight of at least one amphoteric surfactant other than amidopropyl betaines, relative to the total weight of the composition, and
at least one cosmetically acceptable excipient,
said composition comprising at least 65% by weight of water relative to the total weight of the composition, the content of said mixture of surfactants being between 5 and 18% by weight relative to the total weight of the composition, and said composition being free of amphoteric surfactants from the class of amidopropyl betaines and surfactants comprising at least one sulfate group.

2. The transparent cosmetic composition according to claim 1, wherein the total content of anionic surfactant selected in the group consisting of alaninates, acyl glutamic acids and the cosmetically acceptable salts thereof is between 1.5 and 5% by weight, relative to the total weight of the composition.

3. The transparent cosmetic composition according to claim 1, wherein the content of lauryl glucoside as sole non-ionic surfactant of the composition is between 3 and 10% by weight, relative to the total weight of the composition.

4. The transparent cosmetic composition according to claim 1, wherein said anionic surfactant selected from alaninates is selected in the group consisting of sodium cocoyl alaninate and trisodium dicarboxymethyl alaninate.

5. The transparent cosmetic composition according to claim 1, wherein said at least one anionic surfactant selected in the group consisting of acyl glutamic acids and the cosmetically acceptable salts thereof is selected in the group consisting of acyl glutamic acids in which the acyl group comprises a saturated or unsaturated, linear or branched hydrocarbon chain, of 8 to 30 carbon atoms.

6. The transparent cosmetic composition according to claim 1, wherein said at least one amphoteric surfactant is selected in the group consisting of coco-betaine, lauryl betaine, sodium cocoamphoacetate and disodium cocoamphodiacetate.

7. The transparent cosmetic composition according to claim 1, wherein said at least one cosmetically acceptable excipient is selected in the group consisting of emollients, conditioners, solubilizers, perfumes, and the mixtures thereof.

8. The transparent cosmetic composition according to claim 1, wherein said composition is free of thickening agents.

9. The transparent cosmetic composition according to claim 1, comprising at least one conditioner selected in the group consisting of guar, polyquaternium, polyglyceryl, polysaccharides and squalane.

10. The transparent cosmetic composition according to claim 9, wherein the total content of conditioner(s) is between 0.1 and 1% by weight, relative to the total weight of the composition.

11. The transparent cosmetic composition according to claim 1, comprising at least one cosmetically active ingredient selected in the group consisting of vitamins, hyaluronic acid, oat sap, helychrysum extracts, helychrysum essential oils, limnanthes oil, in particular Limnanthes alba seed oil, oat seedlings, flower acids, Garcinia extracts, milk thistle oil, niacinamide, Edelweiss extracts, quinquina extracts, caffeine, peony extracts, mango butter and citron extracts.

12. The transparent cosmetic composition according to claim 1, packaged in the form of shower gel, shampoo, face and/or body foaming gel.

13. The transparent cosmetic composition according to claim 1, said transparent composition having a viscosity greater than 800 cPs.

14. A non-therapeutic cosmetic method for washing and/or care of the skin and/or hair, comprising topically applying to the skin and/or hair a transparent cosmetic composition according to claim 1.

15. The cosmetic method according to claim 14, wherein said skin is sensitive skin.

16. A non-therapeutic cosmetic treatment method for skin and/or hair hygiene and/or care, by means of a transparent cosmetic composition according to claim 1, comprising the consecutive steps consisting of:
applying said transparent cosmetic composition on the skin and/or hair,
developing foam by rubbing the composition on the skin and/or the hair,
and rinsing.

17. The transparent cosmetic composition according to claim 1, having a viscosity of between 800 and 5500 cPs.

18. The treatment method of claim 16, wherein said skin and/or hair have been made wet before applying said transparent cosmetic composition thereon.
